(19) United States
(12) Patent Application Publication
(10) Pub. No.: US 2017/0119300 A1
(43) Pub. Date: May 4, 2017

(54) Sensor Management And Record Tracking System

(71) Applicant: Patrona Medical, Inc., Greenville, SC (US)

(72) Inventor: James E. Conner, Landrum, SC (US)

(73) Assignee: Patrona Medical, Inc., Greenville, SC (US)

(21) Appl. No.: 15/333,495

(22) Filed: Oct. 25, 2016

Related U.S. Application Data
(60) Provisional application No. 62/247,767, filed on Oct. 29, 2015.

(51) Int. Cl. A61B 5/20; A61B 10/00; A61B 5/00; A61B 5/1455; G01J 3/50; A61M 25/00; A61M 39/24; G01N 21/78; G01F 23/68; G01F 23/296; G01N 33/493; A61B 5/145

(52) U.S. Cl. CPC A61B 5/208; G01N 33/493; A61B 10/007; A61B 5/002; A61B 5/7282; A61B 5/746; A61B 5/1455; A61B 5/14546; A61M 25/0017; A61M 39/24; G01N 21/78; G01F 23/68; G01F 23/2962; G01J 3/50; A61B 2562/0295; G01N 2021/7759

(57) ABSTRACT

The present invention is an early infection detection system comprising: a housing having a sensor system section and tray section; an inflow port in fluid communications with a catheter to allow bodily fluid to flow from a patient into the tray section; a color sensor included in the sensor section and in electrical communications with a control module configured to sense the color of a reagent and convert the color to a numeric value representing the amount of a compound, such as bacteria, present in the bodily fluid; a tray carrying a reagent that darkens relative to the amount of the compound present in a bodily fluid that contacts the strip; and, computer readable instructions included in the control module for receiving the numeric value and transmitting the numeric value to a local server.

### BACKGROUND OF THE INVENTION

1) Field of the Invention

The present disclosure is directed in general to wireless or wired monitoring systems that assist with the prediction and notifications of medical staff or other caregivers or individuals of potential complications related to infections, wound treatment, invasive surgery, urinary tract complications, heart catheterizations, pacemaker installation, and other postsurgical procedures or medical monitoring situations.

2) Description of Related Art

An expanding and aging population is placing unprecedented strain on health care systems and its practitioners. The medical community, not unlike other service-providers, is under increasing pressure to serve its growing customer base quickly, efficiently, and cost-effectively while simultaneously minimizing incidents that can be life-threatening.

Although many medical procedures and treatments, for instance, have become nearly routine, even minor complications from these common procedures can easily exceed ten thousand dollars per patient in additional costs.

In October 2014, the Centers for Medicare & Medicaid Services (CMS) began reducing Medicare payments for “subsection (d)” hospitals that rank in the worst performing quartile of subsection (d) hospitals with respect to hospital-acquired conditions (HACs). The worst performing quartile is identified by calculating a Total HAC score which is based on the hospital's performance on 3 quality measures (patient safety indicator 90 composite, central-line associated bloodstream infection, and catheter associated urinary tract infection). Hospitals with a Total HAC score above the 75th percentile of the Total HAC Score distribution may be subject to payment reduction

Urinary tract infections (UTIs) are tied with pneumonia as the second most common type of healthcare-associated infection, second only to surgical site infections (SSIs) and account for more than 15% of infections reported by acute care hospitals are reported on www.medicare.gov. The majority of healthcare-associated UTIs are caused by instrumentation of the urinary tract. More specifically, although it is possible to acquire a UTI in a non-hospital setting, Catheter-Associated Urinary Tract Infections (CAUTIs) are due to the insertion of a Foley catheter in a hospital setting.

CAUTIs can lead to complications such as prostatitis, epididymitis, and orchitis in males, and cystitis, pyelonephritis, gram-negative bacteremia, endocarditis, vertebral osteomyelitis, septic arthritis, endophthalmitis, and meningitis in all patients. Complications associated with CAUTIs cause discomfort to the patient, prolonged hospital stay, and increased cost and mortality as reported on www.medicare.gov. Each year, more than 13,000 estimated deaths are associated with UTIs. Therefore, finding a way of reducing CAUTIs and other post-surgical complications is imperative.

Earlier predictions and alerts to CAUTIs that may ensue from urology procedures could reduce the severity of complications, or prevent them, and lower costs. Currently, CAUTIs are discovered usually after multiple days, not within minutes or hours of a growing colony count. For example, a patient in an inpatient unit has a Foley catheter inserted. The following day is the date of event for a CAUTI. Because the catheter has not been in place for over 2 calendar days from the date of event, it is not classified as a CAUTI. However, depending on the date of admission, this may be a healthcare-associated UTI. Therefore, a Foley catheter, for instance, may not be removed from the patient and antibiotics administered until an infection becomes dangerously symptomatic.

What is needed in the medical and healthcare industry is a system that: detects postoperative complications in areas that include urology, cardiology, and other fields based on automatic, continuous monitoring without human intervention or manual tracking, observation, and testing; provides real-time early warning of symptoms on-set from any communications device; provides summary and detail reports on a patient or an entire patient population; and provides an ability to remotely monitor patients during a hospital stay and after discharge from a hospital.

It is an object of the present invention to provide information to care provides to assist in the early detection of UT's so it can be determined whether patients being admitted have a UTI or develop a UTI after admission.

It is an object of the present invention to provide for a system and method that lower health risks and reduce costs and provide a higher quality of life for patients by early on-set detection of post-surgical complications.

### SUMMARY OF THE INVENTION

The above objectives are accomplished according to the present invention by providing an early infection detection system comprising: a housing having a sensor system section and tray section; an inflow port in fluid communications with a catheter, such as a Foley catheter, to allow bodily fluid to flow from a patient into the tray section; a color sensor included in the sensor section and in electrical communications with a control module configured to sense the color of a reagent and convert the color to a numeric value representing the amount of a compound present in the bodily fluid; a tray carrying a reagent that darkens relative to the amount of the compound present in a bodily fluid that contacts the strip; and, computer readable instructions included in the control module for receiving the numeric value and transmitting the numeric value to a local server.

The invention can include a urine meter in fluid communications with the tray section. The urine meter can be in fluid communications with the tray section allowing bodily fluid to flow from the tray section into the urine meter. The invention can include a first section and a second section defined in the urine meter wherein the first section and the second section are in fluid communications; and, a fluid level assembly for detecting the volume of bodily fluid in the urine meter included in the urine meter. An output valve can be in fluid communications with the urine meter allowing bodily fluid to be removed from the urine meter. A second color sensor can be included in the sensor section. A second reagent can be carried by the tray that darkens relative to the amount of a second compound contained in the bodily fluid; and, the second color sensor can detect the color of the second reagent and transmits a numeric value representing the colony count to the control module. The first compound can react to nitrites and the second compound can reacts to leukocytes. Compounds can also be used for detection of other items beyond nitrites and leukocytes.

According to embodiments of the invention, sensors are programmed to test for multiple conditions in addition to CAUTIs. For instance, the presence of specific compounds in urine may be used to determine the approximate values of the various analytes. Reactions between chemicals on pads or filters and compounds in urine, combined with certain color indicators, will produce colors that correlate with the presence and concentration of the relevant analyte. For instance, an RGB sensor or any sensor that is configured to measure color or the transition or change in color, according to the disclosure may be used to determine health conditions including but not limited to the values shown in the following Table 1.

TABLE 1
EXPECTED VALUES/SENSITIVITY:

| | |
|---|---|
| BLD: | No blood in normal urine (0.015 mg/dl sensitivity) |
| KET: | No ketones in normal urine (5-10 mg/dl sensitivity) |
| GLU: | Concentrations as little 0.1 g/dl may be significantly |
| | abnormal if found consistently (100 mg/dl |
| | sensitivity) |
| RO: | 1-14 mg/dl may be excreted by the normal kidney |
| | (15 mg/dl sensitivity) |
| pH: | Newborn: 5.0-7.0; Thereafter: 4.5-8.0; Average: 6.0 |
| MICROALBUM1N: | Normal albumin levels in random urine are under 20 |
| | mg/L |
| CREATININE: | Creatinine is usually present in random urine in |
| | levels between 10-300 mg/dl |

According to another embodiment of the present disclosure, a system for predicting an infection may include a sensor configured to detect a health metric, an application server configured to manage digital health content by: adding the sensor to a server monitoring queue; base lining the sensor; setting a sensing time limit for the sensor; wherein the sensor is in electronic communication with the application server and the health metric is sensed by the sensor; and the health metric is assessed by the application server to be a predictor of a health complication; and configuring the health metric to form a configured health complication so that the health complication is displayed on a communications device.

The sensor may be a color sensor such as Red-Green-Blue sensor, a liquid volume sensor, a blood detection sensor, a pressure sensor and other detectors. In one embodiment, the sensor RGB color sensor can include physical properties or components taken from the group of true 8-bit RGB format; light intensity sensor, CIE color sensing, integrated proximity sensor, automatic color matching, interrupt output pin on color match, built in white light illuminator, dimmable 0-100%, reads color in just 400 ms, narrow 15° half angle sensing area, a housing is made of 304 stainless steel, water proof/dust proof case, weight of about weight 145 g, and any combination thereof.

The health metric may be urine volume, a change in litmus paper color based on the presence of nitrites, leukocytes or other compounds. In still other embodiments, the metric may include glucose, bilirubin, specific gravity, blood, proteins, acidity, bacteria, swelling, blood pressure, heart rate, and the like.

The health metric may be assessed at least twice to negate a false positive, and the time limit is about 90 seconds to about 120 seconds. These times can be configurable by the care giver. More specifically, reaction time for nitrite and leukocytes is approximately 90 seconds while other substances can have varied reaction times. Thus, the timed interval can be “user defined” and the reaction time is not limited to the foregoing example. For instance, the time range could be defined as being at or between about 1-1800 seconds (30 minutes).

In another aspect of the disclosure, a system for predicting an infection may include a urine sensor; an application configured to manage digital health data; wherein the urine sensor includes a filter, a color sensor, and a volume sensor, the color sensor in communication with the application and configured to test a flow of urine at least twice, from 90-120 seconds, and to sense a change in color in the filter imperceptible to the naked eye, and if a color change is indicated, the application being configured to send an alert to an electronic device.

In a further aspect of the disclosure, a sensor and animating code detect color variations in litmus strips from urine contact imperceptible to the human eye to detect in near real-time an array of potential medical conditions before infections and other complications develop.

According to other aspects of the disclosure, certain embodiments may communicate via various wireless protocols, such as ZigBee™ protocol. Though not limited to a ZigBee™ communication system, ZigBee™ is a wireless, open global standard that supports low-power wireless M2M networks. The ZigBee™ standard operates on an IEEE 802.15.4 physical radio specification and in unlicensed bands including 2.4 GHz, 900 MHz, and 868 MHz. Additionally, or alternatively, other embodiments of the disclosure may utilize Bluetooth™ devices operating at 2.4 GHz in the license-free, globally available Industrial, Scientific, and Medical (ISM) radio band. The advantage of operating in this band is worldwide availability and compatibility.

Additional objects and advantages of the present subject matter are set forth in, or will be apparent to, those of ordinary skill in the art from the description herein. Also, it should be further appreciated that modifications and variations to the specifically illustrated, referenced, and discussed features, processes, and elements hereof may be practiced in various embodiments and uses of the disclosure without departing from the spirit and scope of the subject matter. Variations may include, but are not limited to, substitution of equivalent means, features, or steps for those illustrated, referenced, or discussed, and the functional, operational, or positional reversal of various parts, features, steps, or the like. Those of ordinary skill in the art will better appreciate the features and aspects of the various embodiments, and others, upon review of the remainder of the specification.

### BRIEF DESCRIPTION OF THE DRAWINGS

A full and enabling disclosure of the present subject matter, including the best mode thereof, directed to one of ordinary skill in the art, is set forth in the specification, which makes reference to the appended figures, in which:

FIG. 1 is a perspective view of aspects of the invention and an enlarged portion of aspects of the invention;

FIG. 2 shows a side view of aspects of the invention;

FIGS. 3A and 3B show top down views of aspects of the invention;

FIG. 3C shows a side view of aspects of the invention;

FIG. 3D shows a side view of aspects of the invention; and,

FIG. 4 is a top view of aspects of the invention.

It will be understood by those skilled in the art that one or more aspects of this invention can meet certain objectives, while one or more other aspects can meet certain other objectives. Each objective may not apply equally, in all its respects, to every aspect of this invention. As such, the preceding objects can be viewed in the alternative with respect to any one aspect of this invention. These and other objects and features of the invention will become more fully apparent when the following detailed description is read in conjunction with the accompanying figures and examples. However, it is to be understood that both the foregoing summary of the invention and the following detailed description are of a preferred embodiment and not restrictive of the invention or other alternate embodiments of the invention. In particular, while the invention is described herein with reference to a number of specific embodiments, it will be appreciated that the description is illustrative of the invention and is not constructed as limiting of the invention. Various modifications and applications may occur to those who are skilled in the art, without departing from the spirit and the scope of the invention, as described by the appended claims. Likewise, other objects, features, benefits, and advantages of the present invention will be apparent from this summary and certain embodiments described below, and will be readily apparent to those skilled in the art. Such objects, features, benefits, and advantages will be apparent from the above in conjunction with the accompanying examples, data, figures, and all reasonable inferences to be drawn therefrom, alone or with consideration of the references incorporated herein.

### DETAILED DESCRIPTION OF A PREFERRED EMBODIMENT

With reference to the drawings, the invention will now be described in more detail. Unless defined otherwise, all technical and scientific terms used herein have the same meaning as commonly understood to one of ordinary skill in the art to which the presently disclosed subject matter belongs. Although any methods, devices, and materials similar or equivalent to those described herein can be used in the practice or testing of the presently disclosed subject matter, representative methods, devices, and materials are herein described.

As required, detailed embodiments are disclosed herein; however, it is to be understood that the disclosed embodiments are merely exemplary and may be embodied in various forms. Therefore, specific structural and functional details disclosed herein are not to be interpreted as limiting, but merely as a basis for the claims and as a representative basis for teaching one skilled in the art to variously employ the exemplary embodiments of the present disclosure, as well as their equivalents.

In general, the present disclosure provides systems and methods for monitoring patient metrics and predicting complications from medical procedures before they arise and wirelessly transmitting predictors to caretakers. For instance, in the fields of cardiology and urology, some systems disclosed herein predict a UTI before the UTI develops. Other systems according to the disclosure monitor and electronically communicate patient health metrics predicting other health conditions and events associated with post-surgical procedures or invasive treatments, or provide monitoring and alerts to caregivers during a hospital stay or in a patient's home, such as by sending reminders regarding drug administration schedules and dosages.

Unless defined otherwise, all technical and scientific terms used herein have the same meaning as is commonly understood by one of ordinary skill in the art to which this disclosure belongs. In the event that there is a plurality of definitions for a term or acronym herein, those in this section prevail unless stated otherwise:

An application-programming interface (API), which is a set of programming instructions and standards for accessing a software application via the Internet. Typically, a software company will publish or release its API to the public for other software developers to design products to exploit the software application.

Within the context of computer hardware and software, an application program is a set of one or more computer programs that performs a function when executed within a computer hardware device. If the set is comprised of multiple programs, the programs are coordinated to perform a function together but such programs may individually perform other functions. Similarly, a program may be comprised of multiple modules that perform certain functions individually and other functions when combined in various ways.

Communications device(s). Any electronic device having a screen and an interactive interface such as a keyboard or touchpad to enable a user to receive or send messages. Optionally, the screen also serves as the input device, and is a touch screen. Examples include but are not limited to: telephones such as smart phones, desktop computers, laptops, tablets, pads, vehicle systems, smart appliances, and the like.

In one embodiment, a UTI can be defined as when a colony count is greater than 100,000 bacteria. For a UTI, E. coli accounts for about 50% of UTI cases. The gram-negative species Klebsiella, Proteus, Enterobacter, Pseudomonas, and Serratia account for about 40%, and the gram-positive bacterial cocci, E. faecalis, S. saprophytics, and Staphylococcus aureus account for the remainder.

Detailed reference will now be made to the drawings in which examples embodying the present subject matter are shown. The detailed description uses numerical and letter designations to refer to features of the drawings. The drawings and detailed description provide a full and written description of the present subject matter, and of the manner and process of making and using various exemplary embodiments, so as to enable one skilled in the pertinent art to make and use them, as well as the best mode of carrying out the exemplary embodiments. The drawings are not necessarily to scale, and some features may be exaggerated to show details of particular components. Thus, the examples set forth in the drawings and detailed descriptions are provided by way of explanation only and are not meant as limitations of the disclosure. The present subject matter thus includes any modifications and variations of the following examples as come within the scope of the appended claims and their equivalents.

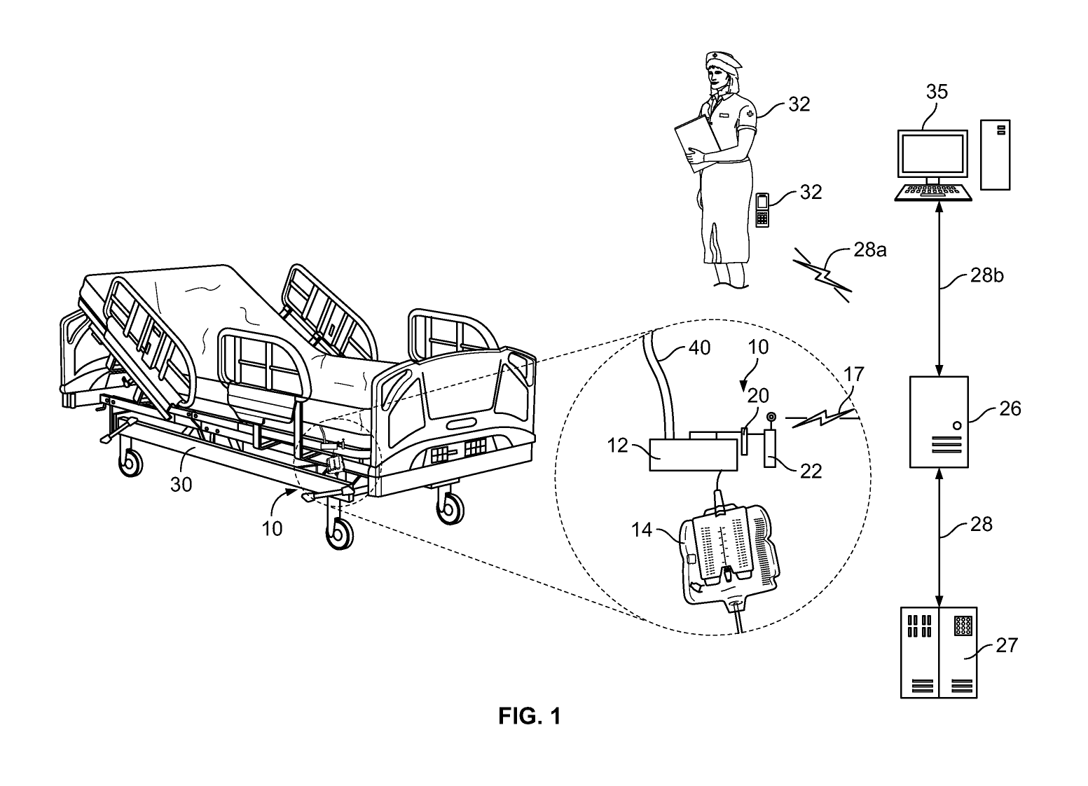

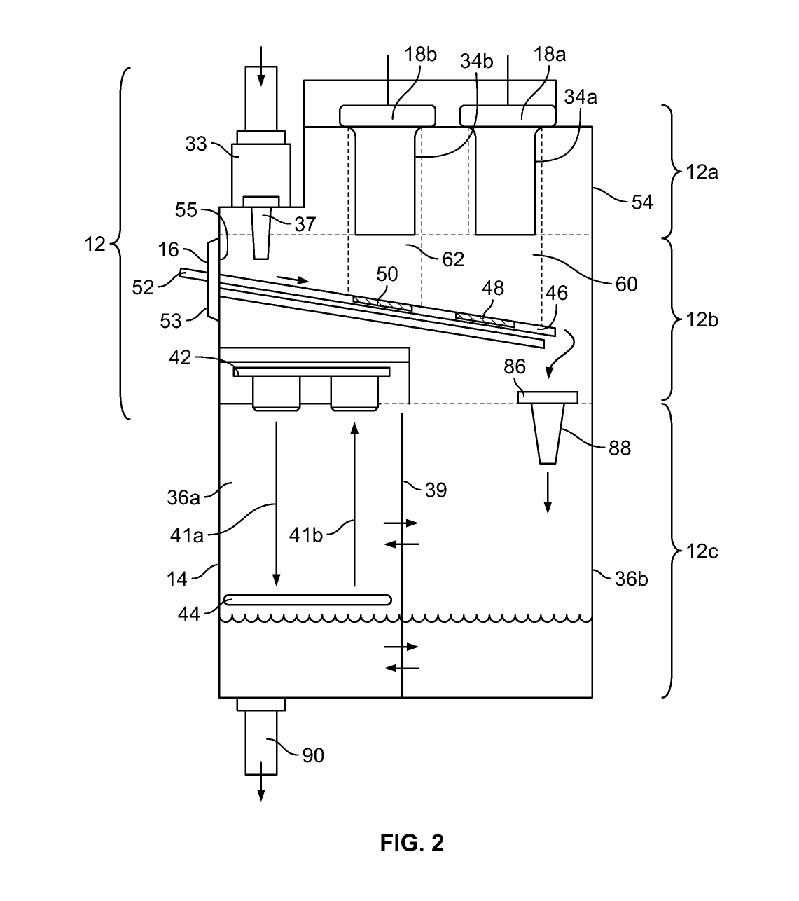

Turning now to FIG. 1, a urological or urinary monitoring or tracking and alert system according to an aspect of the disclosure is designated in general by the numeral 10. The system 10 broadly includes a UTI sensor system 12 located in a sensor section 12a, a tray assembly located in a tray section 12b and a urine meter or volume sensor 14 located in a urine meter section 12c. The urine meter can be in communications with the sensor system or directly attached to the sensor system as shown in FIG. 2. The sensor system can include or be attached to a control module 20 and a gateway repeater 22. The control module can include a software application that, though a sensor system communications link 18, can be in communications with a local server system 26. The local server can be in communications with a wireless, cellular, satellite network 28a or direct network 28b. A cable harness 31 can be used to provide electronic communications from sensors in the sensor system 12 to the control module 20 or gateway repeater 22. The communications can be wired or wireless.

The sensor can send numerical values representing colony counts to the control module which can then send the numeric value to the local server. In one embodiment, the control module is programmable to send a notification to the local server of the numeric value exceeds a predetermined value representing a colony count associated with a UTI In one embodiment, the control module sends the numeric value to the local server and the local server generate an alert based upon predetermined criteria so that the local server can send an appropriate alert to a caregiver.

Here, for convenience and practicality, portions of the system 10, such as the urine volume sensor 14, may be positioned in close proximity to a patient's bed 30.

More specifically, the UTI sensor system 12 generally will include the urine volume sensor 14, a urine reagent tray, disperser or filter 16 (FIG. 2), and the sensor 18a and 18b arranged to both transmit a patient's urine production and to predict a UTI before one develops. The production of urine is a valuable tool to understand the health and well-being of a patient, and the software according to the present disclosure causes urine data to be recorded and entered automatically in a patient's Electronic Health Record (EHR) and to provide summary and detail reports regarding the patient or an entire patient population. Further discussion of the exemplary operation and additional details of the software is found below.

The software application is compatible with various operating systems (OS) of personal computers (PC), laptops, notepads, and is available in a mobile application for smart phones. A Windows© OS version is available in a Cloud-based platform and a version is available for installation on a local workstation or server. The Windows® application 24, for example, may have some or all of the exemplary features shown in Table 2.

TABLE 2
Monitor patient population or a single patient Alerts to potential
Urinary Tract Infection: Audible alert Text Message Email Message
Visual Warning Light Alerts Ability to detect life cycle of urine:
Patient information entered by keyboard or scanner device: Ability
to load floor plan coordinated with sensor identification.

The repeater 22 may be multiple repeaters placed at multiple preferred locations throughout a hospital on multiple floors to provide line of sight to allow for continuous connectivity between the control module 20 and the local server 26. One or more repeaters can be directly connected to the control module and wireless connected to additional repeaters. In one embodiment, the control module includes a transceiver that can wireless communicate with one or more repeaters. The local server 26 can be in communications with remote server 27 allowing for an aggregation of data and remote storage of data along the medical information of the patient to be access at different health care provider locations.

A caregiver such as a nurse 32 can place a Foley catheter in the urinary tract of a patient. As the patient voids, urine may enter the urine meter 14 through first line 40. The system 10 can analyze the urine to provide analytical results allowing a care giver to predict a UTI and alert the caregiver 32 over the network 28a to remove the catheter before an infection develops. The caregiver can be notified by mobile device 32 that can be carried by the caregiver or can be notified through workstation 35 or other display apparatus such as multi user monitor or display.

Referring to FIG. 2, the sensors 18a and 18b can be received into sensor cavities 34a and 34b allowing the sensors to be recessed into the sensor section 12a. A cap can be placed above the sensors to assist in the protection of the sensors as well as assist with preventing ambient light from interfering with the sensors operation. The sensors can convert color information detected by the sensor such as RGB values and convert these to frequencies. The values can be compared to a preexisting database of known values. The database can be derived from actual experimentation and clinical tests of subjects with known conditions. The frequency can be a square wave in one embodiment. Various functionality of the sensors can be modified through programming of the sensor by the control module. For example, resolution, scan rate, and sensitivity can be modified. The sensors can be connected to the control module through a direct connection such as wires or a wiring harness.

The volume of urine entering the urine meter 14 from the catheter in communication with the patient may be determined by a fluid level assembly that can include a sensor 42, such as an ultrasound or capacitive sensor. In one embodiment, the sensor 42 is an ultrasound or ultrasonic sensor and utilizes a float 44 placed inside the urine meter 14, which transmits a volume signal 41a resulting in a reflection 41b back to the sensor 42 to gauge distance. The sensor 42, in electronic communications with the control module, sends the urine volume data to the control module 20. The control module can send the data to the gateway 22, which is transmitted to the server system 26. Data handling and analysis according to the disclosure are controlled by the software application and transmission may be wireless or by cellular or satellite, or by direct network and other transmission means according to network or communications 17 or 28. In one embodiment, the ultrasound sensor 42 is not disposable and will separate from the urine meter 14 upon removal of the Foley catheter.

In one embodiment, the ultrasonic sensor sends a brief chirp with an ultrasonic speaker and makes it possible for the integrated circuit associated with the ultrasonic sensor to measure the time it takes the echo to return to its ultrasonic microphone after rebounding from the float. The chirp can be a 40 kHz tone. When the ultrasonic sensor detects the echo with its ultrasonic microphone, it changes that high signal back too low. The sensor's integrated circuit then determines and stores how long the high signal from the sensor lasted in a variable. The time measurement is how long it took sound to travel to the object and back. With this measurement, you can then use the speed of sound in air to calculate the distance in centimeters, inches, feet, volume, etc. of the float from the sensor thereby determining the fluid volume in the urine meter.

Once urine flows into the tray assembly disposed in a tray section 12b, the urine can then flow into the urine meter 14 through fluid meter opening 86. Fluid meter opening 86 can include a fluid meter one way valve 88 preventing fluid from back flowing from the urine meter into the tray assembly if the orientation of the invention is changed through movement or other cause. In one embodiment, the urine meter can include first compartment 36a and second compartment 36b separated by divider 39. The divider can include an opening in the divider to allow fluid communications between the first and second compartment. The first compartment can include a fluid level assembly that can include a sensor and float.

The urine meter can be emptied through output valve 90 in the urine meter and can be disposed. To reduce the changes of the spread of infection, the urine meter can be attached to a sealed waste container and include a back flow valve between the urine meter and waste container so that back flow from waste container into the urine meter are minimized. The output valve can be configured to open automatically based in response to a predetermined total volume of the container of the urine meter. The fluid can flow from the urine meter container to a waste system to reduce the transmission of in infection by reducing of eliminating the care giver need to empty the container. The waste system can be emptied or otherwise managed by an individual that is not the care giver or in direct contact with the patient.

As introduced above, the UTI sensor system 12 may include a sensor system disposed in the sensor section 12a. A tray assembly can include a tray opening 55 in the tray section 12b that can be attached to the sensor section. A tray 16 can be received in the opening. The urinary tract sensors 18a and 18b can provide information from the reagents on the tray to the sensors to assist in the prediction of a UTI before one develops. Here, the exemplary tray 16 may be constructed of three layers 46, 48, and 50. The first layer 46 may be a plastic backing, the second layer 48 may be a nitrite strip, and the third layer 50 may be a leukocyte strip. The plastic backing 46 may range from 1 mil (2.5-6.0 millimeters) to any suitable thickness to provide adequate rigidness to support the litmus paper sections 48 and 50. The nitrite or second strip 48 may be of paper construction with 5-70% cotton fiber impregnated with a reagent that is marked to detect traces of nitrite at (0.075 mg/dl sensitivity). The leukocyte or third strip 50 may be constructed in similar fashion and can detect traces of leukocytes at 10-15 WBC/μL sensitivity. These strips 48, 50 and/or other strips can test urine for various bio markers such as blood sugar, ketones, pH, and the like.

In one embodiment, the widths and lengths of the strips 46, 48, and 50 may be approximately ½ inch (1.27 cm) wide and approximately ½ inch (1.27 cm) in length, or other widths or lengths may be used to provide adequate coverage for the sensors 18a and 18b. In this example, the lengths of the nitrite strip 50 will be relatively shorter to allow sensor access to the leukocyte strip 48. Additionally, as explained by exemplary operation below, the strips 48 and 50 will change color based on the percentage or quantity of compounds that can represent the amount of bacteria found in bodily fluid such as urine. In one embodiment, suitable papers for uses as strips 48 and 50 are available from Tcco Diagnostics of Anaheim, Calif., by way or one non-limited exemplary size and substance. For instance, other monitoring systems according to the disclosure may be used to detect a complication other than a CAUTI and could employ filters in multiple configurations. The dimensions of the filters could range, for instance, in size from 0.5 mm in width to approximately 0.5 mm in length or greater than 0.5 mm. Also, appropriate sensitivities of strips will be adjusted to detect conditions other than leukocyte and nitrite as shown in one embodiment in Table 3.

TABLE 3

| | |
|---|---|
| BLD: | No blood in normal urine (0.015 mg/dl sensitivity) |
| K.ET: | No ketones in normal urine (5-10 mg/dl sensitivity) |
| GLU: | Concentrations as little 0.1 g/dl may be significantly |
| | abnormal if found consistently (100 mg/dl |
| | sensitivity) |
| PRO: | 1-14 mg/dl may be excreted by the normal kidney |
| | (15 mg/dl sensitivity) |
| pH: | Newborn: 5.0-7.0; Thereafter: 4.5-8.0; Average: 6.0 |
| MICROALBUMIN: | Normal albumin levels in random urine are under 20 |
| | mg/L |
| CREATININE: | Creatinine is usually present in random urine in |
| | levels between 10-300 mg/dl |

The tray 16 may be replaced during the average shift change of a caregiver; for example in 8 or 12 hour time intervals or at the discretion of the healthcare provider. Accordingly, the tray 16 may include a handle or gripping portion 52 affixed to the layers 46, 48, and 50 to allow for easy handling, insertion, and removal by caregivers. A hilt 53 can be included with the tray to cover opening 55 in the tray assembly to prevent or reduce urine that may escape through the opening in the tray assembly.

The urinary tract sensor assembly 12a, which works in conjunction with the tray assembly and tray section and its reagents 48 and 50, may be RGB sensor, and may include a housing 54 made of black plastic or other similarly durable, dark material. Although schematically shown square in shape, the housing 54 may be tubular or other shape. With some sensors, RGB sensitivity is affected by ambient light where the sensor system is located; therefore, the housing 54 is constructed to block ambient light from entering the housing 54 or tray assembly.

The urinary tract sensor system may be attached to the Foley catheter via an inflow port 33 that can be a quick connect device or other suitable attachment. The sensor system may include at least two sensors 18a and 18b, each having respective, separate chambers 60 and 62 in one embodiment. The chambers can include side walls that extend down from the sensors to the tray and can be in close proximity to the tray to asset with blocking ambient light from the sensor and reagent. In one embodiment, sensors include a light source and the chambers can keep the light generated from the sensors from overflowing into the neighboring sensors chamber.

Urine can enter the tray section from the patient at inflow port 33. A inflow one-way valve 37 can prevent urine or other fluids from flowing back into the line connected to the quick connect regardless of the orientation of the sensor system. For example, if the sensor system is moved, rotated or otherwise orientated, the one-way valve prevents retrograde flow/backflow.

The urine can be channeled across the tray where sensors 56 and 58 read their respective reagents such as litmus strips 48 and 50 are cooperatively associated to register the RGB values or other color designations and measurements such as CMYK, wavelengths or other measurements. In one embodiment, a colony counts for leukocytes and nitrites greater than 100,000 bacteria indicate a UTI. The reagents can react with urine within or about 90 seconds after urine makes contact with the reagents so that should any bacteria be present (for example, a colony count greater than 10,000), the reagent can begin to turn color invisible to the naked eye but which the RGB sensor 18a or 18b can recognize as a change or increase in value from the last value measured and transmit the results or trigger an alert to a caregiver. In one embodiment, the sensors 18a and 18b can determine if the urine sample falls within one of the following colony count ranges: less than 10,000; 10,000-15,000, 15,000-20,000, increasing by 5,000 and continuing to and in excess of a 100,000 count. Other ranges between 0 and 100,000 can be used. In one embodiment, the upper limits can exceed 100,000. Notifications or alerts can be generated when the colony count exceed one of these ranges, and/or increasing over time or moves from one range to another. In one embodiment, the sensor can color match the strip or reagent to a known color value allowing increased sensitivity and resolution when determining if the color falls within a range associated with colony count.

In one embodiment, the control module can alert the caregiver once the colony count exceed 100,000 and such alert will not be removed unless manually cleared through the control module. Notably, after 90-100 seconds, reagents such as litmus strips will continue to migrate towards a positive color match due to continued contact with the urine. Since the values will continue to rise (e.g., value>5 up to 20), the system is programmed to ignore values after completion of a timed interval, such as after 110 seconds. If a predetermined threshold is exceeded, a positive match is indicated and healthcare staff will install new reagents or a new tray with new reagents according to a removal and installation procedure described below. The software will recognize the new filter 16 because the new color value is base lined for a dry filter (e.g., value=5) will be detected signifying a reset of the sensor system.

When the patient voids again, the cycle as described above can repeat. A second positive match will result in a positive prediction of a UTI and protocol will require the staff to remove the Foley catheter and administer antibiotics before a UTI can develop. In one embodiment, the actual colony count ranges are less important and may be eliminated. In this embodiment, the control module uses an initial colony count as a baseline. A second colony count as a predetermined interval is made and compared to the baseline. If the colony count is rising, a notification can be made indicating the potential existence of a UTI. The notification can also be triggered when the difference between the baseline and the second reading exceed a predetermined value such as 10,000 in a predetermined period of time, such as 60 minutes. In one embodiment, a second colony count can be made. The notification can be triggered when the difference between the first colony count and the baseline is positive and the difference between the second colony count and the first colony count is positive.

The notification may be transmitted by electronic mail, text message, audio or the like via local server 26 or remote server 27 to any electronic device. After the foregoing real-time analysis, the urine is then channeled from the sensor system and tray assembly to a urine meter assembly 14 for measuring the associated volume as described above.

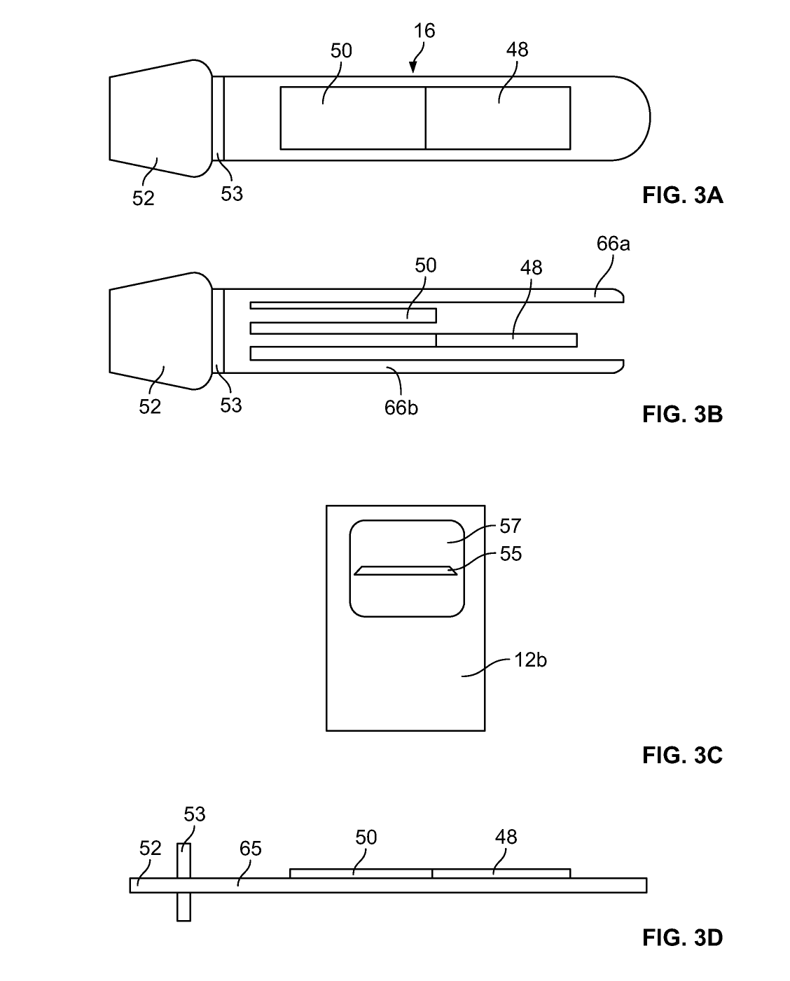

Referring to FIG. 3A, the tray 16 may be a disposable or consumable item to be replaced by the healthcare provider according to local protocol. The filter 16 may be handled by the gripping portion 52 to slide in and out of the tray section via a rail system 66a and 66b and tray opening 55 (FIG. 3C) in the tray section 12b. The tray can include rails disposed laterally on the tray that are received in slots disposed in the tray section. The tray opening 55 may receive the tray. The tray opening can be defined in the tray section as shown in 3C. A gasket 53 may be provided at the filter opening 66 to create an air and water tight connection. In one embodiment, the tray includes a substrate 65 and the reagents are support by the substrate.

A UTI occurs over a series of days and not minutes or hours. This means that there is no presence of bacteria upon the initial installation of the tray, but, for instance, 24 hours later the sensors begin to register an increasing value as expected. Once two positive alerts are achieved, the Foley catheter is removed, and the patient is given antibiotics, thus preventing a potential infection before a colony count reaches a diagnostic level.

In one embodiment a light or color sensor is used. The sensor includes a light source and a color detection sensor such as an RGB sensor. The sensor assembly may include separate sensors 56 and 58 to monitor red, green and blue color levels of respective, separate strips 48 and 50 of the tray 16. The reagents will reflect the bacteria count represented by color variations and the RGB sensor can gauge the color variations and produce a numeric value for each change in value.

In one embodiment, a magneto potentiometer sensor may be used as a urine volume sensor. More particularly, the potentiometer sensor may have a disposable and non-disposable component. The disposable component is a magnet. The non-disposable component can be a sensor device that registers height of the magnet. In this example, the magnet is pressed into a lightweight float and as the float height increases based on an increase in the volume of the urine, the sensor registers this change and transmits the data to the software. The software will convert analog data into a value that represents the urine volume in milliliters.

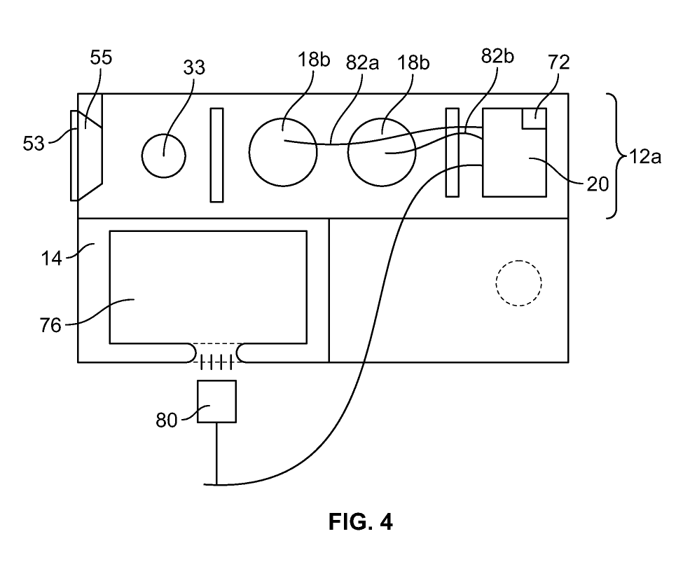

Referring to FIG. 4, a top down view, an ultrasound sensor 76 may be used in the urine volume sensor 14 in one embodiment. The ultrasound sensor 76 may be positioned atop the urine meter 14 and may be disposable to be discarded with the urine meter upon removal from patient. The ultrasound sensor 76 detects the height of a fluid table and transmits the amount of time lapse from signal transmit to signal receive. The software can convert this data into volume in milliliters. The ultrasound sensor 76 can communicate the data to the gateway 22 via the control module 20. In one embodiment, the control module 20 is reusable and is not discarded after use. The control module 20 may contain a programmable circuit board, a communication board, a power supply, and a cable system to connect to various sensors. For instance, in one embodiment, a suitable circuit board may be an Arduino™ Uno™ board with the software embedded in Java® language, for example, and the circuit board will serve as the control unit for the sensors. The control module 20 may be powered with a replaceable power source such as a 9 volt battery, or it could employ another power source and is modular to adapt to multiple sensor capabilities. The control module 20 can include an onboard wireless and or cellular transmitter and receiver 72 capabilities for dependability and flexibility. The disclosure is not limited to the foregoing examples and may employ other microcontrollers such Raspberry PI™ or Beagle Bone™ and the like. Likewise, the software is not limited to Java® or a particular language and could employ other languages C+, C#, Python, and the like.

The volume sensor can be connected to a wiring harness that can be connected to the control module. The sensors can be connected to the control module through wires 82a and 82b. From this top down view, the width of the sensor system can be less than that of the urine meter.

In one embodiment, a capacitive liquid level sensor may be used to determine the level of urine produced by a patient.

In one embodiment, a health complication or infection sensor system can be included in the sensor system The sensor system may be attached to a Foley catheter via a quick disconnect device or other suitable attachment. The assembly may include at least two sensors, each having respective, separate chambers. Here, urine may enter the assembly from a patient to be channeled across a tray where the sensors can read their respective reagents such as litmus strips and register RGB or other color values.

The sensor system may include other sensors each having respective, separate chambers. Here, the presence of specific compounds in the urine may be used to determine approximate values of various analytes whereby reactions between chemicals on reagent, pas or strips and the compounds in the urine, combined with certain color indicators, will produce colors that correlate with the presence and concentration of the relevant analyte and thus provide predictors of health complications. In this example, the sensors can detect blood on the pad or reagent. Similarly, the sensors may detect glucose on the pad or reagent. Exemplary values and sensitivities of the invention include but are not limited to those shown in Table 4.

TABLE 4
EXPECTED VALUES/SENSITIVITY:

| | |
|---|---|
| BLD: | No blood in normal urine (0.015 mg/dl sensitivity) |
| KET: | No ketones in normal urine (5-10 mg/dl sensitivity) |
| GLU: | Concentrations as little 0.1 g/dl may be significantly |
| | abnormal if found consistently (100 mg/dl |
| | sensitivity) |
| PRO: | 1-14 mg/dl may be excreted by the normal kidney |
| | (15 mg/dl sensitivity) |
| pH: | Newborn: 5.0-7.0; Thereafter: 4.5-8.0; Average: 6.0 |
| MICROALBUMIN: | Normal albumin levels in random urine are under 20 |
| | mg/L |
| CREATININE: | Creatinine is usually present in random urine in |
| | levels between 10-300 mg/dl |

Other incremental tests and sensitivities relative to the presence of other compounds, chemicals, and concentrations can be set for the system to assist with the predication and diagnosis by caregivers of the existence of onset of other developing health conditions.

The following laboratory trials show exemplary operations of various aspects of the disclosure as discussed above.

This first test utilized an ultrasonic or ultrasound sensor available from Virtuabotix™ as one of the sensor 76. The test criteria and metrics are shown in Table 5.

TABLE 5

| | |
|---|---|
| Working Voltage: | 5 V (DC) |
| Static current: | Less than 2 mA |
| Sensor Angle: | 15 to 35 Degrees |
| Detection Distance: | 2 cm to 500 cm |
| Precision: | Accurate to 0.3 cm |

The second test of urine level sensing utilized a capacitive sensor. The test criteria and metrics are shown in Table 6.

TABLE 6

| | |
|---|---|
| Output: variable resistance: | 400-2000 ohms +/− 20-% |
| Resistance gradient: | 150 ohms/inch (60 ohms/cm) |
| Reference Resistance: | 2000 ohms +/− 20-% |
| Resolution: | <0.01 in (0.25 mm) |
| Power rating: | 0.5 watts (VMax = 10 V) |

The tape sensor detected the fluid level to be 11 cm, which equated to 2 cm accuracy.

This test utilized an RGB sensor available from Virtuabotix™ as one sensor. The alpha test criteria and metrics are shown in Table 7.

TABLE 7

| | |
|---|---|
| Working Voltage: | 3.3 V or 5.0 V DC |
| Photo-diodes: | 3 color photo-diodes (Red, Green, and Blue) |
| Frequencies: | 3 operational frequencies 2%, 20% |
| | (recommended and 100%) Recommended |
| Focal Distance: | 1 to 5 inches (2.54 cm to 12.7 cm) |

In one test, the sensor was utilized to test color changes in filter paper used as the reagents or pad. Here, an Arduino® board setup was employed, and the RGB sensor was shielded at from surrounding light sources to achieve an undiluted base line.

Table 8, below, shows average results for the RGB values for different shades of the purple litmus paper strips measured by the RGB sensor placed 3 cm from the litmus paper. The percentage values at the top of each group indicate the shade of purple of each of the litmus paper detected with 0% being the lightest while the color darkens as the percentage increases representing an increase in bacteria in a fluid contacting the litmus paper.

TABLE 8

| 0% | | | 20% | | | 40% | | | 60% | | | 80% | | | 100% | | |
|---|---|---|---|---|---|---|---|---|---|---|---|---|---|---|---|---|---|
| R | G | B | R | G | B | R | G | B | R | G | B | R | G | B | R | G | B |
| 253 | 252 | 253 | 252 | 252 | 258 | 251 | 251 | 232 | 249 | 248 | 251 | 247 | 245 | 250 | 246 | 243 | 249 |

Table 9, below, shows results for the RGB values for different shades of the purple litmus paper strips measured by the RGB sensor placed 1 cm from the litmus paper.

TABLE 9

| 0% | | | 20% | | | 40% | | | 60% | | | 80% | | | 100% | | |
|---|---|---|---|---|---|---|---|---|---|---|---|---|---|---|---|---|---|
| R | G | B | R | G | B | R | G | B | R | G | B | R | G | B | R | G | B |
| 243 | 239 | 242 | 240 | 235 | 239 | 237 | 230 | 237 | 227 | 213 | 228 | 218 | 201 | 223 | 208 | 181 | 211 |

The distance between the sensor and the tray can be in the range of 0.5 cm to 15 cm in one embodiment. The set of RGB values generated from the output of the sensor is dependent on the distance between the sensor and the litmus paper as well as the present of lack of ambient light. Accordingly, care should be taken to block the ambient light such as using a black material around the sensor to shield it from surrounding light sources.

The software is a monitoring and notification software application that integrates all deployed medical sensors. The software provides to an administrator a graphical user interface (GUI) that enables seamless addition, management, or deletion of medical sensors from the wireless network.

The software can allow all sensors in the network to be displayed. An administrator can add sensors as they are deployed and can also delete any sensors that are out of commission.

A screen or display can be used to show readings received from the RGB or other color sensors and displayed for review. In one embodiment, the detection of the color of a litmus paper can be shown. A screen area above a chart area can display a rendition of the color of the litmus paper. Should the detected color exceed a set threshold, it can be flagged for notification to the user. Should there be a positive match on two RGB sensors where one is monitoring for nitrite and the other is monitoring for leukocytes, as described hereinabove, then the software can send a notification to hospital staff to check the patient to prevent an infection.

The sensor devices interface with the software application to provide user-friendly graphical user interface (GUI). The sensors can be connected to a computer running the software using a USB cable or wirelessly. By way of example and not of limitation, XBee radios may be utilized to achieve wireless connectivity between sensors and any computer running the software. The radios may be configured using, for example, XCTU™ software from Digi® International.

What is claimed is:

1. An early infection detection system comprising:
a housing having a sensor system section, tray section, and volume section;
an inflow port in fluid communications with a catheter to allow bodily fluid to flow from a patient into the tray section;
an inflow one way valve configured to prevent bodily fluid from flowing from the tray section toward the catheter once the fluid is present in the tray section;
a color sensor included in the sensor section and in electrical communications with a control module configured to sense the color of a reagent and convert the color to a numeric value representing the amount of a compound present in the bodily fluid;
a chamber extended into the tray section and receiving the sensor for blocking ambient light;
a tray carrying a reagent that darkens relative to the number of bacteria present in a bodily fluid that contacts the strip;
a urine meter opening in fluid communications with the tray section and the urine meter allowing bodily fluid to flow from the tray section into the urine meter;
a urine meter one way valve configured to prevent bodily fluid from flowing from the urine meter into the tray assembly;
a first section and a second section defined in the urine meter wherein the first section and the second section in fluid communications with each other;
a fluid level assembly for detecting the volume of bodily fluid in the urine meter;
an output valve in fluid communications with the urine meter allowing bodily fluid to be removed from the urine meter; and,
computer readable instructions included in the control module for receiving the numeric value from the sensor, determining if the numeric value exceeds a predetermined number, transmitting a notification to a local server that is configured to transmit an alert to a caregiver.

2. The system of claim 1 including a desktop computer in communication with the local server for receiving the alert.

3. The system of claim 1 wherein the notification is generated when the colony count exceeds 100,000.

4. The system of claim 1 including a ultrasonic sensor including in the fluid level assembly cooperatively associated with a float disposed in the first section of the urine meter.

5. The system of claim 1 including a reagent carried by tray that will react to the presents of a substance selected from the group consisting of: nitrites, leukocytes, ketones, albumin, glucose, blood, proteins and any combination thereof.

6. The system of claim 1 including a reagent carried by tray that will react to the presents of a substance selected from the group consisting of: nitrites, leukocytes, and any combination thereof.

7. The system of claim 1 including a light source included in the sensor.

8. The system of claim 1 including:
a second color sensor included in the sensor section;
a second reagent carried by the tray that darkens relative to the amount of nitrites contained in the bodily fluid; and,
wherein the second color sensor detects the color of the second reagent and transmits a numeric value representing the colony count to the control module.

9. The system of claim 1 including lateral rails included in the tray cooperatively associated with slots in the tray section for supporting the tray and reagents in the tray section a predetermined distance under the sensors.

10. The system of claim 9 wherein the predetermine distance is in the range of 0.5 cm to 15 cm.

11. The system of claim 1 included a gasket attached to the tray to assist in the prevention of bodily fluid from escaping the opening in the tray section that receive the tray.

12. An early infection detection system comprising:
a housing having a sensor system section and tray section;
an inflow port in fluid communications with a catheter to allow bodily fluid to flow from a patient into the tray section;
a color sensor included in the sensor section and in electrical communications with a control module configured to sense the color of a reagent and convert the color to a numeric value representing the amount of a compound present in the bodily fluid;
a tray carrying a reagent that darkens relative to the amount of the compound present in a bodily fluid that contacts the strip; and,
computer readable instructions included in the control module for receiving the numeric value and transmitting the numeric value to a local server.

13. The system of claim 12 including a urine meter in fluid communications with the tray section.

14. The system of claim 12 including a urine meter in fluid communications with the tray section allowing bodily fluid to flow from the tray section into the urine meter.

15. The system of claim 12 including:
a first section and a second section defined in the urine meter wherein the first section and the second section are in fluid communications; and,
a fluid level assembly for detecting the volume of bodily fluid in the urine meter included in the urine meter.

16. The system of claim 15 including an output valve in fluid communications with the urine meter allowing bodily fluid to be removed from the urine meter.

17. The system of claim 12 including:
a second color sensor included in the sensor section;
a second reagent carried by the tray that darkens relative to the amount of a second compound contained in the bodily fluid; and,
wherein the second color sensor detects the color of the second reagent and transmits a numeric value representing the colony count to the control module.

18. The system of claim 17 wherein the first compound reacts to nitrites and the second compound reacts to leukocytes.

19. An early infection detection system comprising:
a housing;
an inflow port in fluid communications with a catheter to allow bodily fluid to flow from a patient into the housing;
a first color sensor attached to the housing and in electrical communications with a control module configured to sense the color of a reagent and convert the color to a numeric value representing the amount of a compound present in the bodily fluid;
a second color sensor attached to the housing and in electrical communications with the control module configured to sense the color of a second reagent and convert the color to a second numeric value representing the amount of a second compound present in the bodily fluid;
a tray carrying the first reagent and second reagent receiving by the housing and configured to dispose the first reagent under the first sensor and the second reagent under the second sensor; and,
computer readable instructions included in the control module for receiving the first numeric value and the second numeric value and transmitting the numeric values to a local server.

20. The system of claim 19 wherein the local server includes server computer readable instructions for converting the number values to bacteria colony counts and transmitting an alert to a caregiver is the colony count exceed a predetermined number.
